(19) United States
(12) Patent Application Publication
(10) Pub. No.: US 2009/0171175 A1
(43) Pub. Date: Jul. 2, 2009

(54) Personalized Medical Monitoring: Auto-Configuration Using Patient Record Information

(75) Inventors: Li Li, Milpitas, CA (US); Scott Amundson, Oakland, CA (US); Corydon A. Hinton, Oakland, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/343,733

(22) Filed: Dec. 24, 2008

Related U.S. Application Data
(60) Provisional application No. 61/009,682, filed on Dec. 31, 2007.

(51) Int. Cl. A61B 5/1455

(52) U.S. Cl. 600/324

(57) ABSTRACT

According to various embodiments, methods and systems are provided for configuring a medical device connected to a network based on an identification. In one embodiment, a method is provided that includes receiving an identification, retrieving configuration parameters from a network based at least in part upon the identification, and selecting one or more configuration parameters based at least in part upon the identification. In various embodiments, a system is provided that includes a medical device configured to communicate over a network, wherein the medical device may receive information from the network and the monitor is configured to select one or more configuration parameters based at least in part upon the information received from the network.

### RELATED APPLICATION

This application claims priority to U.S. Provisional Application No. 61/009,682, filed Dec. 31, 2007, and is incorporated herein by reference in its entirety

### BACKGROUND

The present disclosure relates generally to medical devices and, more particularly, to operation and configuration of such medical devices.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring physiological characteristics. Such devices provide caregivers, such as doctors, nurses, and/or other healthcare personnel, with the information they need to provide the best possible healthcare for their patients. As a result, such monitoring devices have become an indispensable part of modern medicine. For example, one technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Pulse oximeters and other medical devices are typically positioned around a patient's bed or around an operating room table. When a caregiver desires to operate the medical device (e.g., program, configure, and so-forth) they manipulate controls or push buttons on the monitoring device itself. For each individual patient or setting, the medical device may need to be configured to work optimally with the patient or setting. Additionally, different caregivers, e.g., nurses, doctors, etc, may need access to different configuration settings on the medical device.

This conventional configuration, however, may have several issues. First, as described above, the medical device must be configured to work with a specific patient. For example, in the case of a pulse oximeter, different alarms and/or sampling rates may be configured depending on the condition of the patient. If a caregiver wishes to configure the medical device to reflect new or updated patient information, he or she may need to review the appropriate medical records and enter the new or updated configuration information. Additionally, the configuration of medical device may depend on the location device. For example, a patient monitor located in an intensive care unit may require different configuration parameters as compared to a patient monitor located in a sleep unit. Also, as many medical devices are mobile and often moved from one patient room or hospital area to another, the medical devices may need to be configured again in the new room or location. Further, relying on a caregiver to enter information from medical records, patient charts, etc., may introduce an element of human error to the configuration of such medical devices. If a caregiver mistypes or misreads a configuration setting, the patient monitor or other medical device may not be configured correctly, resulting in incorrect readings, inaccurate patient monitoring, etc.

Second, as discussed above, different caregivers may have different roles in configuring a medical device. For example, a nurse's task may be to simply make minor adjustments depending on the patient's status over a short period. In contrast, a physician may desire to make additional configuration changes to a medical device based on a larger review of a patient's medical history, the patient's current status, etc. Thus, it may be against hospital policy for a nurse or other caregiver to make changes to those configuration parameters adjusted by a physician or doctor.

### SUMMARY

Certain embodiments commensurate in scope with the disclosure are set forth below. It should be understood that these aspects are presented merely to provide the reader with a brief summary of certain forms of the disclosure might take and that these aspects are not intended to limit the scope of the disclosure. Indeed, the disclosure may encompass a variety of aspects that may not be set forth below.

In one embodiment a method of operating a medical device is provided that includes receiving an identification, retrieving information from a network based on the identification, and selecting one or more configuration parameters based on the identification.

An embodiment of a method of operating a medical device is provided that includes receiving an identification number, retrieving information from a memory based on the identification number, and selecting one or more configuration parameters based on the identification number.

In another embodiment, a system is provided that includes a medical device configured to communicate over a network, wherein the medical device may receive information from the network and the monitor is configured to select one or more configuration parameters based on information received from the network.

In another embodiment, a tangible machine-readable medium is provided that includes code configured to retrieve information from a network based on an identification provided to a medical device.

### BRIEF DESCRIPTION OF THE DRAWINGS

Advantages of the disclosure may become apparent upon reading the following detailed description and upon reference to the drawings in which:

FIG. 1 illustrates a pulse oximetry system coupled to a multi-parameter patient monitor and a sensor according to various embodiments;

FIG. 2 illustrates multiple pulse oximetry systems coupled to a network in accordance with an embodiment;

FIG. 3 is a block diagram of a sensor and monitor in accordance with an embodiment;

FIG. 4 is flowchart of a process for configuring a monitor in accordance with an embodiment; and

FIG. 5 is a flowchart of a process for configuring a monitor in accordance with another embodiment.

### DETAILED DESCRIPTION OF SPECIFIC EMBODIMENTS

Various embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Various embodiments may provide a medical device capable of retrieving information and configuring itself according to the retrieved information. The configuration may be automated or may involve interfacing with a caregiver to select one or more configuration parameters. The information may be retrieved or the configuration may be based on various parameters, including a patient ID, a caregiver ID, a caregiver's permission level, the location of the medical device, etc.

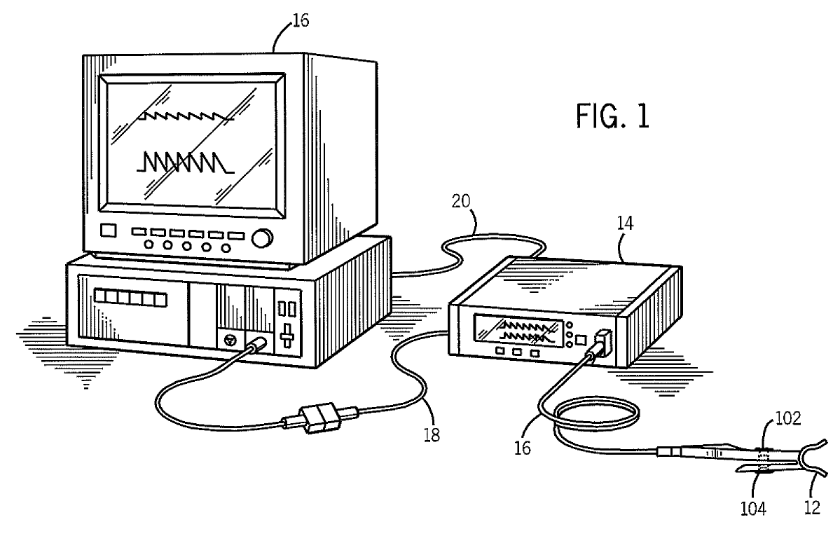

Referring now to FIG. 1, an embodiment of a medical monitoring system is depicted that includes a sensor 12 used in conjunction with a patient monitor 14. In the depicted embodiment, a sensor cable 16 connects the sensor 12 to the patient monitor 14. As will be appreciated by those of ordinary skill in the art, the sensor 12 and/or the sensor cable 16 may include or incorporate one or more integrated circuit devices or electrical devices, such as a memory, processor chip, or resistor, that may facilitate or enhance communication between the sensor 12 and the patient monitor 14. Likewise the sensor cable 16 may be an adaptor cable, with or without an integrated circuit or electrical device, for facilitating communication between the sensor 12 and various types of monitors, including older or newer versions of the patient monitor 14 or other physiological monitors. As will be appreciated, the sensor cable 16 may be used to transmit control and/or timing signals from the monitor 14 to the sensor 12 and/or to transmit acquired data from the sensor 12 to the monitor 14.

In an embodiment, the patient monitor 14 may be a suitable pulse oximeter, such as those available from Nellcor Puritan Bennett LLC. In other embodiments, the patient monitor 14 may be a monitor suitable for measuring tissue water fractions, or other body fluid related metrics, using spectrophotometric or other techniques. Furthermore, the monitor 14 may be a multi-purpose monitor suitable for performing pulse oximetry and measurement of tissue water fraction, or other combinations of physiological and/or biochemical monitoring processes, using data acquired via the sensor 12. Furthermore, to upgrade conventional monitoring functions provided by the monitor 14 to provide additional functions, the patient monitor 14 may be coupled to a multi-parameter patient monitor 16 via a cable 18 connected to a sensor input port and/or via a cable 20 connected to a digital communication port, for example.

In an embodiment, the sensor 12, in the example depicted in FIG. 1, is a clip-style sensor that includes an emitter 22 and a detector 24 which may be of any type suitable for spectrophotometric measurement. For example, the emitter 22 may be one or more light emitting diodes adapted to transmit one or more wavelengths of light, such as in the red to infrared range, and the detector 24 may be a photodetector, such as a silicon photodiode package, selected to receive light in the range emitted from the emitter 22. Suitable sensors are available from Nellcor Puritan Bennett LLC, for example. In the depicted embodiment, the sensor 12 is coupled to a sensor cable 16 that is responsible for transmitting electrical and/or optical signals to and from the emitter 22 and detector 24 of the sensor 12. The sensor cable 16 may be permanently coupled to the sensor 12, or it may be removably coupled to the sensor 12—the latter alternative being more useful and cost efficient in situations where the sensor 12 is disposable. In alternate embodiments, the sensor 12 may take other suitable forms besides the form illustrated in FIG. 1. For example, the sensor 12 may be configured to be clipped onto a finger or earlobe or may be configured to be secured with tape or another static mounting technique.

In various embodiments, the patient monitor 14 may include any number of selectable configuration parameters to facilitate the operation of the monitor 14. For example, the monitor 14 may include configuration of alarm thresholds, sampling rates, patient history, etc. A caregiver may desire to adjust the configuration parameters based on the status of the patient, the patient's characteristics such as weight, age, etc, or any other suitable condition. To operate the monitor 14, the monitor 14 may receive and validate the caregiver's ID, and/or the patient's ID, or another suitable ID or access mechanism. As described further below, various embodiments of the monitor 14 may retrieve information from a memory of the monitor, or a network coupled to the monitor, and automatically or semi-automatically configure the parameters based on the patient ID, caregiver ID, and/or other retrieved information.

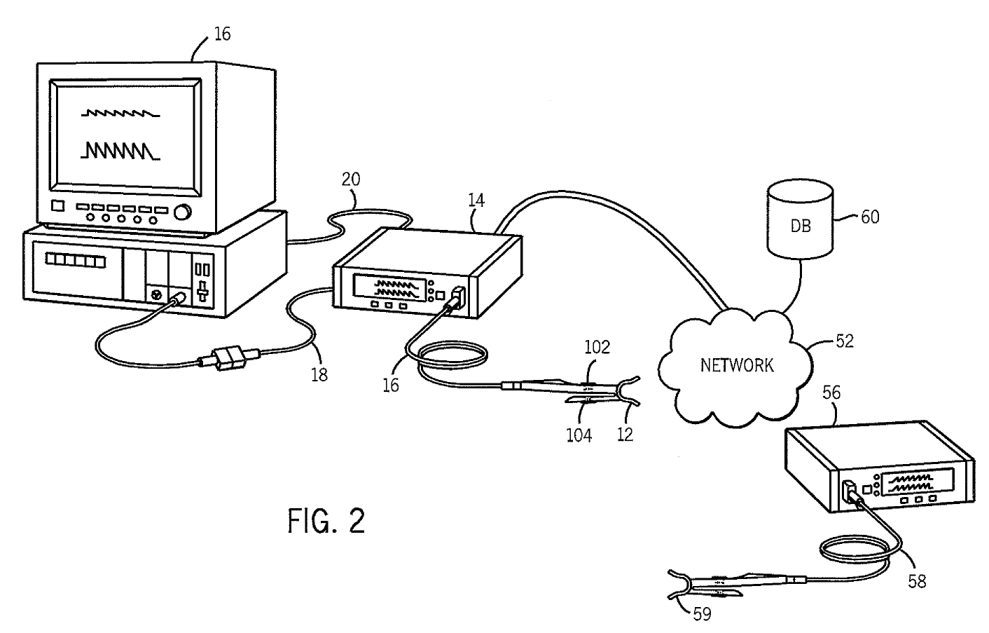

FIG. 2 is an illustration of an embodiment of a system 50 that includes the sensor 12, the patient monitor 14, and a multi-parameter patient monitor 16. In this embodiment, the patient monitor 14 may be connected to a network 52, such as a network in a hospital or other medical facility. The patient monitor 14 may be connected to the network via a physical connection, such as network cable, or through wireless communication technology, such as Wi-Fi, WiMax, Bluetooth, IR, etc. The network 52 may be a LAN, WAN, MAN, and may use any suitable network technology such as Ethernet, wireless Ethernet, etc, or any combination thereof.

In various embodiments, additional devices may be connected to the network, such as another monitor 56. The monitor 56 may be connected to another sensor 59 located in another area of the hospital or medical facility, such as by a sensor cable 58. Additionally, a patient database 60 may be coupled to the network 52. The database 60 may store patient medical records that include patient history, patient status, patient treatment, etc. Further, the database 60 may store previous configurations of the monitor 14, monitor 56, or any other medical device that may be used, even if the medical device is not connected to the network 50.

In various embodiments, a medical device, such as the monitor 16 or monitor 56, may retrieve information over the network and use it to configure the parameters of the monitor 14 or monitor 56. For example, upon activating the monitor 14, such as by turning on the monitor and entering or scanning a caregiver or patient ID, the monitor 14 may contact the database 60 and provide the caregiver ID and/or patient ID. The database 60 may then retrieve records and/or configuration parameters associated with the caregiver ID and/or patient ID and provide them to the monitor 14. Similarly, the monitor 56 or other medical devices may also retrieve information from the database.

In other embodiments, the monitor 14 or other medical devices may retrieve information from a memory located in the monitor 14, monitor 56, or the sensor 12. For example, the memory may be a non-volatile memory, such as flash, that may store information about the patient, the medical device, etc. Once the medical device, e.g. the monitor 14, is activated, the monitor may check the memory for information related to an entered caregiver ID and/or patient ID. Thus, the memory may store information about the caregiver or patient, such as patient status, patient characteristics, clinical orders, history patient data, etc. In other embodiments, a medical device, such as the monitor 14 or the monitor 54, may retrieve information over a network and from a memory located in another medical device or other connected device. In such an embodiment, the medical device may compare the information received over the network and use the information that is most recently updated to configure itself.

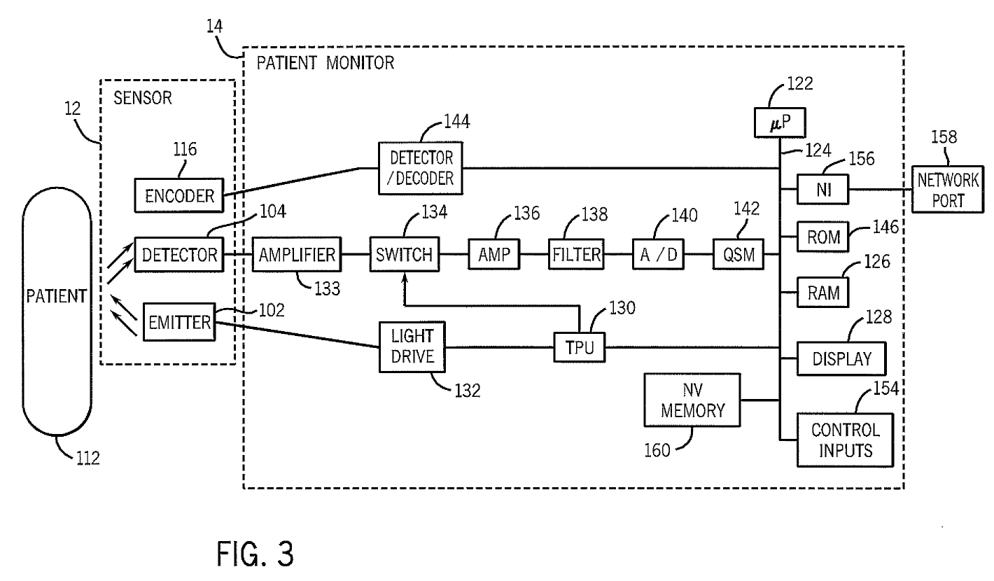

FIG. 3 is a block diagram of an embodiment of a patient monitor 14 that may be configured to implement the techniques described herein. In an embodiment, the patient monitor 14 may be a suitable pulse oximeter, such as those available from Nellcor Puritan Bennett LLC. Light from an emitter 102 may pass into a patient 112 and be scattered and detected by a detector 104. The sensor 12 is connected to a patient monitor 14. The monitor 14 may include a microprocessor 122 connected to an internal bus 124. Also connected to the bus 124 may be a RAM memory 126 and a display 128. A time processing unit (TPU) 130 may provide timing control signals to light drive circuitry 132 which may control when the emitter 102 is illuminated, and if multiple light sources are used, the multiplexed timing for the different light sources, TPU 130 may also control the gating-in of signals from detector 104 through an amplifier 133 and a switching circuit 134. These signals may be sampled at the proper time, depending upon which of multiple light sources is illuminated, if multiple light sources are used. The received signal from the detector 104 and the contact sensor 102 may be passed through an amplifier 136, a low pass filter 138, and an analog-to-digital converter 140. The digital data may then stored in a queued serial module (QSM) 142, for later downloading to RAM 126 as QSM 142 fills up. In one embodiment, there may be multiple parallel paths of separate amplifier, filter and A/D converters for multiple light wavelengths or spectra received.

In an embodiment, the sensor 12 may also contain an encoder 116 that provides signals indicative of the wavelength of one or more light sources of the emitter 102 to allow the monitor 14 to select appropriate calibration coefficients for calculating a physiological parameter such as blood oxygen saturation. The encoder 116 may, for instance, be a coded resistor, EEPROM or other coding devices (such as a capacitor, inductor, PROM, RFID, a barcode, parallel resonant circuits, or a calorimetric indicator) that may provide a signal to the processor 122 related to the characteristics of the sensor 10 that may allow the processor 122 to determine the appropriate calibration characteristics for the sensor 12. Further, the encoder 116 may include encryption coding that prevents a disposable part of the sensor 102 from being recognized by a processor 122 that is not able to decode the encryption.

In various embodiments, based at least in part upon the value of the received signals corresponding to the light received by detector 104, the microprocessor 122 may calculate a physiological parameter using various algorithms. These algorithms may utilize coefficients, which may be empirically determined, corresponding to, for example, the wavelengths of light used. These may be stored in a ROM 146. In a two-wavelength system, the particular set of coefficients chosen for any pair of wavelength spectra may be determined by the value indicated by the encoder 116 corresponding to a particular light source in a particular sensor 10. For example, the first wavelength may be a carbon dioxide signal wavelength, and the second wavelength may be a water correction wavelength. In one embodiment, multiple resistor values may be assigned to select different sets of coefficients. In another embodiment, the same resistors may be used to select from among the coefficients appropriate for an infrared source paired with either a near red source or far red source. The selection between whether the wavelength sets can be selected with a control input from control inputs 154. Control inputs 154 may be, for instance, a switch on the monitor, a keyboard, or a port providing instructions from a remote host computer.

In various embodiments, the monitor 14 may be connected to a network 52, via a network interface 156. The network interface 156 may implement any networking technology or protocol, such as Ethernet, wireless Ethernet, etc. The network interface 156 may be connected to a network port 158, via a network cable, or via a wireless connection if the network interface 156 implements wireless technology. Additionally, the monitor 14 may include a non-volatile memory 160 that may store caregiver preferences, patient information, or any other information useful for configuring the monitor 14. The software for performing the configuration of the monitor 14 and retrieval of information over the network interface 156 may also be stored on the memory 160, or may be stored on the ROM 146.

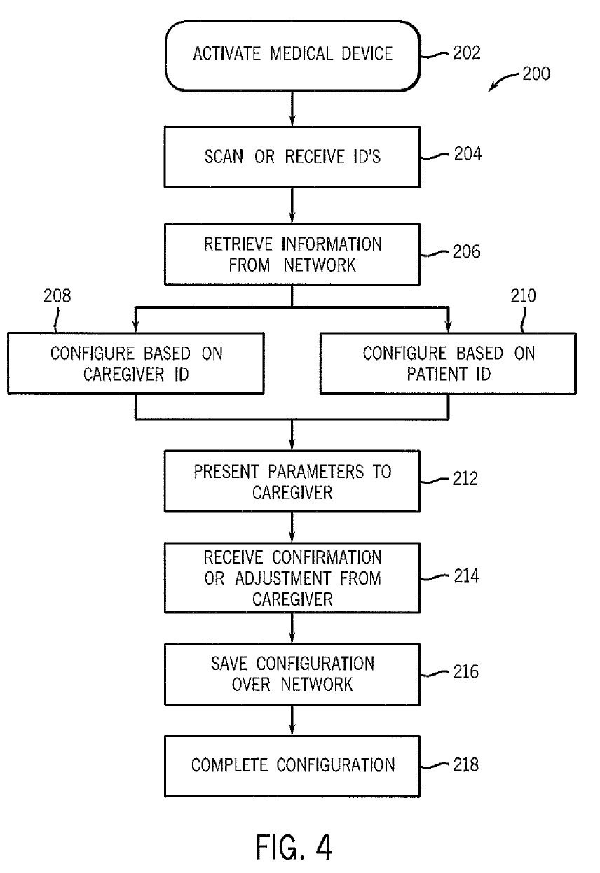

FIG. 4 is a flowchart of an embodiment of a process 200 for configuring a medical device, such as the medical monitor 14. Initially, in this embodiment, the monitor may be activated (block 202) such as by turning on, bringing out of suspend, connecting a sensor, etc. The caregiver operating the monitor may scan or enter the appropriate identifications (block 204). For example, the caregiver may scan the patient's ID and the caregiver's ID. As discussed above, in one embodiment the ID's may be entered manually via a keyboard or other input. In other embodiments, the ID information may be incorporated into a barcode and scanned via a bar code reader, or the ID may include an RFID and the medical device may include an RFID reader.

In various embodiments, once the medical device has received the ID's for the patient and/or the caregiver, the medical device may retrieve information from its memory or over the network based on those ID's (block 206). For example, as discussed above, the medical device may contact a database over a network. In one embodiment, the medical device itself may execute the software necessary to retrieve records from a database based on the caregiver ID and patient ID. In other embodiments, the software for retrieving the records from the database may be executed on the database server. In yet other embodiments, any number of databases, other servers, or any other suitable device may be connected to the network and accessed by the medical device.

In various embodiments, the medical device may configure various parameters based on the caregiver's ID (block 208). For example, specific preferences for the caregiver, such as display options, refresh rates, etc., may be retrieved and configured in the monitor. Similarly, the parameters may also be configured based on the caregiver's status or permission levels. A doctor operating the medical device may have different or more detailed configuration parameters available than a nurse operating the medical device. The medical device may also configure various parameters based on the patient's ID (block 210). For example, electronic medical records (EMR) retrieved from the database may include the patient's status, the patient's characteristics such as weight, height, age, clinical orders, etc.

In various embodiments, once the configuration parameters are selected based on the caregiver's ID, the patient's ID, or both, the selected parameters may be presented to the caregiver via a display or other interface included with or attached to the medical device (block 212). The caregiver may review the configuration and make any adjustments necessary. For example, the caregiver may decide to adjust a parameter based on a recent evaluation of a patient's condition which has not yet been entered into the patient's medical records. The medical device 14 may receive confirmation and/or adjustment of the various parameters (block 214) and then save the configuration (block 216). The medical device 14 may save the configuration to a database 60 or other device on the network 52, so that the configuration may be retrieved for future configuration sessions. After the configuration parameters are saved the configuration is complete and the medical device is ready for use (block 218).

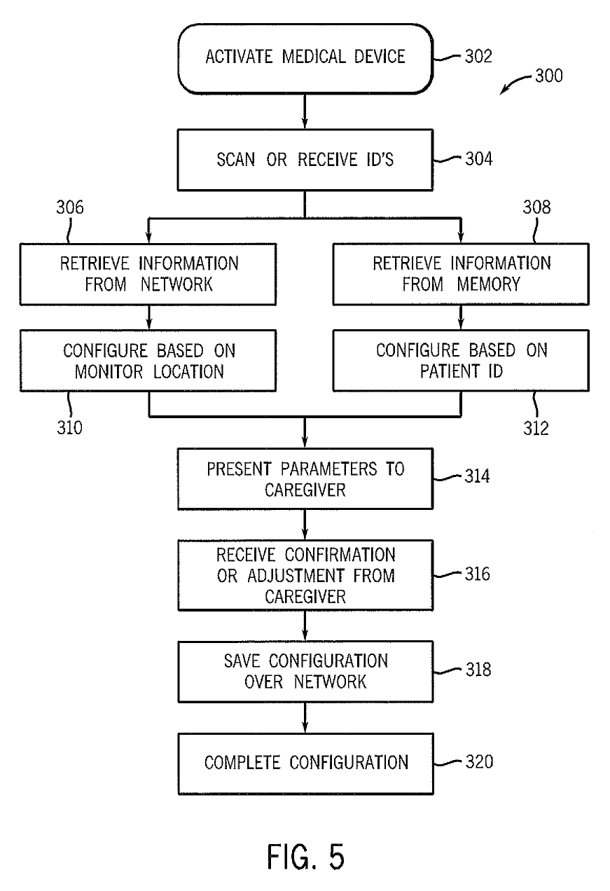

In other embodiments, a medical device, such as the monitor 14, may use more than one technique to set configuration parameters. For example, FIG. 5 depicts a process 300 of an embodiment for configuring a medical device in which configuration information may be stored in memory and on a network. Further, the configuration may be based on additional information, such as the location of the medical device. As discussed above, the medical device, e.g. monitor 14, may be activated (block 302) such as by turning on, awakening from a suspend mode, connecting a sensor, etc. The caregiver's ID and patient's ID may be scanned or entered into the monitor, such as via an input device, bar code scanner, RFID scanner, etc (block 304). The medical device 14 may retrieve information from a network (block 306), such as from a database 60, based on the caregiver's ID and/or patient's ID. Alternatively, or additionally, the medical device 14 may retrieve information from a memory included on the medical device (block 308), such as a solid-state memory or magnetic memory, such as a hard drive.

In an embodiment, the information retrieved over the network 52 may be based on the medical device 14. For example, the medical device 14 may be located on a specific subnet of a TCP/IP network. If the hospital or other medical facility divides different areas into different subnets, the subnet of the medical facility may indicate the location of the monitor 14. In other embodiments, a unique identifier of the monitor, such as a media access control (MAC) address, may be stored in a database and correlated to a location, thus indicating the location of the medical device. Accordingly, if the location of the medical device is determined, the medical device may be configured based on the location of the device (block 310). For example, if the medical device is located in a sleep lab, the medical device may be configured with sleep mode settings, as opposed to a medical device located in neonatal intensive care unit (NICU).

In various embodiments, the medical device may also retrieve information stored on a memory of the medical device, such as previously saved caregiver preferences or patient information. The medical device may also configure the monitor based on the caregiver's ID and/or the patient's ID (block 312) using the information retrieved from memory. In some embodiments, the configuration may be based on both the location of the medical device, the caregiver's ID, the patient's ID, or any combination thereof. Additionally, in some embodiments the medical device may prioritize the configuration selections, such that those configuration selections based on a caregiver's ID have the highest priority, configuration selections based on the medical device's location have the next highest priority, etc.

In various embodiments, once the configuration parameters are selected, they may be presented to the caregiver or user of the medical device (block 314) such that the medical device may receive confirmation of the configuration parameters or adjustment of any parameters the caregiver or user wishes to change (block 316). After the configuration parameters are finalized, the medical device may save the configuration to the memory of the medical device (block 318), thus completing the configuration process (block 320). Future configuration processes may incorporate the changes made by the caregiver by retrieving configuration preferences or other information from the memory.

While the disclosure may be capable of various modifications and alternative forms, various embodiments have been shown by way of example in the drawings and have been described in detail herein. However, it should be understood that the disclosure is not intended to be limited to the particular forms disclosed. Rather, the disclosure is to cover all modifications, equivalents, and alternatives falling within the spirit and scope of the disclosure as defined by the following appended claims.

What is claimed is:

1. A method of operating a medical device, comprising:
receiving an identification;
retrieving configuration parameters from a network based at least in part on the identification; and
selecting one or more configuration parameters based at least in part on the identification.

2. The method of claim 1, wherein the medical device comprises a sensor.

3. The method of claim 2, wherein the sensor comprises a pulse oximetry sensor.

4. The method of claim 1, wherein the medical device comprises a patient monitor.

5. The method of claim 4, wherein the monitor comprises a pulse oximeter.

6. The method of claim 5, wherein the identification comprises a caregiver ID, and/or a patient ID, and/or combinations thereof.

7. The method of claim 5, wherein the identification comprises a location of the medical device.

8. The method of claim 1, wherein the configuration parameters comprise information about a patient.

9. The method of claim 1, comprising saving the selected configuration parameters to a database via the network.

10. A method of operating a medical device, comprising:
receiving an identification;
retrieving configuration parameters from a memory of the medical device based at least in part on the identification; and
selecting one or more configuration parameters based at least in part on the identification.

11. The method of claim 10, comprising displaying the selected one or more configuration parameters on a display of the medical device.

12. The method of claim 10, comprising saving the selected one or more configuration parameters to the memory.

13. The method of claim 10, comprising retrieving information via a network based at least in part on the identification and selecting one or more configuration parameters based at least in part on the identification.

14. The method of claim 10, wherein the identification comprises a caregiver ID, a patient ID, or any combination thereof.

15. The method of claim 10, wherein the identification comprises a location of the medical device.

16. The method of claim 10, wherein the one or more of the configuration parameters comprises information about a patient.

17. A medical device, comprising:
a medical device capable of communicating over a network, wherein the medical device is capable of receiving information from the network, wherein the medical device is capable of selecting one or more configuration parameters based at least in part on the information received from the network.

18. The system of claim 17, wherein the medical device comprises a network interface.

19. The system of claim 17, comprising a database connected to the network.

20. The system of claim 17, comprising a sensor coupled to the monitor and configured to measure a physiological parameter.
